(19) United States
(12) Patent Application Publication
(10) Pub. No.: US 2026/0229002 A1
(43) Pub. Date: Aug. 6, 2026

(54) MEDICAL DEVICE, METHOD, AND STORAGE MEDIUM

(71) Applicant: TERUMO KABUSHIKI KAISHA, Tokyo (JP)

(72) Inventor: Yuki SAKAGUCHI, Fujisawa Kanagawa (JP)

(21) Appl. No.: 19/578,782

(22) Filed: Mar. 25, 2026

Related U.S. Application Data
(63) Continuation of application No. PCT/JP2024/033834, filed on Sep. 24, 2024.

(30) Foreign Application Priority Data
Sep. 25, 2023 (JP) 2023-161617

(51) Int. Cl. G06V 10/25; G06T 7/00; G06T 7/70; G06V 10/26; G06V 10/774; A61B 5/00; G06V 10/82

(52) U.S. Cl. CPC G06V 10/25; G06T 7/0012; G06T 7/70; G06V 10/26; G06V 10/774; A61B 5/0066; G06T 2207/10101; G06T 2207/10132; G06T 2207/20081; G06T 2207/20084; G06T 2207/30052; G06T 2207/30101; G06V 10/82; G06V 2201/03; G06V 2201/07

(57) ABSTRACT

A medical device for determining a boundary in a blood vessel tomographic image, includes a processor configured to perform the steps of: acquiring data indicating a blood vessel tomographic image; executing a call to a machine learning model with the data to detect a first region indicating a detection target and one or more second regions different from the first region, the machine learning model having been trained with: a plurality of training blood vessel tomographic images, and labels indicating positions of first regions and second regions different from the first regions for the training blood vessel tomographic images; determining a boundary of the first region using the detected one or more second regions; and displaying a screen showing the tomographic image with a boundary line corresponding to the determined boundary.

### CROSS-REFERENCE TO RELATED APPLICATION

This application is a continuation of International Patent Application No. PCT/JP2024/033834 filed September 24, 2024, which is based upon and claims the benefit of priority from Japanese Patent Application No.2023-161617, filed September 25, 2023, the entire contents of which are incorporated herein by reference.

### TECHNICAL FIELD

The present disclosure relates to a medical device, a method, and a storage medium.

### Background Art

There is a technology for supporting diagnostic imaging related to vascular treatment such as percutaneous coronary intervention (PCI) using machine learning. For example, there is a known system that performs segmentation of a region of interest using a neural network based on a polar coordinate image obtained by imaging a coronary artery.

### SUMMARY

In one aspect, embodiments of the present disclosure provide a medical device, a method, and a storage medium capable of suitably determining a boundary of a detection target region in a blood vessel tomographic image.

In one aspect, a medical device for determining a boundary in a blood vessel tomographic image, comprises: a display; a memory that stores a program; and a processor configured to execute the program to perform the steps of: acquiring data indicating a blood vessel tomographic image; executing a call to a machine learning model with the data indicating the blood vessel tomographic image to detect a first region indicating a detection target and one or more second regions different from the first region, the machine learning model having been trained with: a plurality of training blood vessel tomographic images, and labels indicating positions of first regions and second regions different from the first regions for the training blood vessel tomographic images; determining a boundary of the first region using the detected one or more second regions; generating a screen showing the blood vessel tomographic image with a boundary line corresponding to the determined boundary; and controlling the display to display the generated screen.

In one aspect, a boundary of a detection target region in a blood vessel tomographic image can be suitably determined.

### BRIEF DESCRIPTION OF DRAWINGS

FIG. 1 is an explanatory diagram illustrating a configuration example of an image diagnosis system.

FIG. 2 is a block diagram illustrating a configuration example of a server.

FIG. 3 is a block diagram illustrating a configuration example of an image diagnosis apparatus.

FIG. 4 is a diagram illustrating a processing example of detecting a detection target region from a blood vessel tomographic image.

FIG. 5 is an explanatory diagram illustrating an outline of a first embodiment.

FIG. 6 is a flowchart illustrating generation processing of a detection model.

FIG. 7 is a flowchart illustrating detection processing of a detection target region.

FIG. 8 is a diagram illustrating an outline of a second embodiment.

FIG. 9 is a diagram illustrating a display example of a detection result of a blood vessel side branch.

FIG. 10 is a flowchart illustrating detection processing of a detection target region according to a second embodiment.

FIG. 11 is a diagram illustrating an outline of a third embodiment.

FIG. 12 is a diagram illustrating a display example of a detection result of a calcified region.

FIG. 13 is a flowchart illustrating detection processing of a detection target region according to the third embodiment.

FIG. 14 is an explanatory diagram illustrating an outline of a modification.

FIG. 15 is an explanatory diagram illustrating an outline of a fourth embodiment.

FIG. 16 is a flowchart illustrating detection processing of a detection target region according to the fourth embodiment.

FIG. 17 is an explanatory diagram illustrating an outline of a fifth embodiment.

FIG. 18 is an explanatory diagram illustrating an outline of the fifth embodiment.

FIG. 19 is an explanatory diagram illustrating a longitudinal tomographic image along a center line of a side branch.

FIG. 20 is a flowchart illustrating display processing of a blood vessel image.

### DETAILED DESCRIPTION

Embodiments of the present disclosure will be described below in detail based on the accompanying drawings.

### First Embodiment

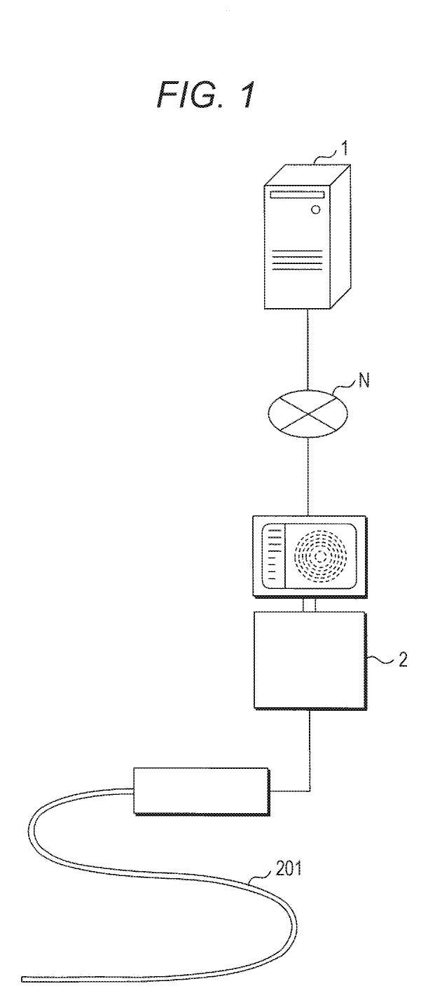

FIG. 1 is a diagram illustrating a configuration example of an image diagnosis system. In the present embodiment, an image diagnosis system that detects a detection target region corresponding to a predetermined detection target (for example, lumen of blood vessel, external elastic membrane, plaque and the like) from a blood vessel tomographic image obtained by imaging a cross section of a patient’s blood vessel will be described. The image diagnosis system includes a server 1 and an image diagnosis apparatus 2. The devices are connected to communicate with each other via a network N.

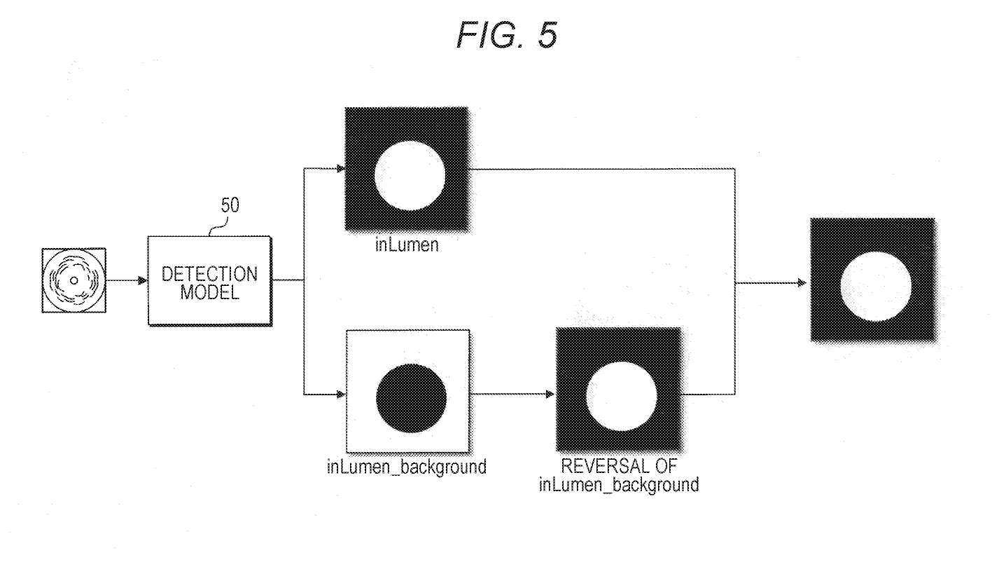

The server 1 is a server computer capable of performing various types of information processing and transmitting and receiving information. Note that a device corresponding to the server 1 may be a device such as a personal computer. The server 1 performs machine learning using predetermined training data to generate a detection model 50 (refer to FIG. 5) that detects a detection target region and a region different from the detection target region using a blood vessel tomographic image as an input.

The image diagnosis apparatus 2 is an imaging apparatus or a medical device that acquires a medical image of a patient’s blood vessel, and has, for example, an intravascular ultrasound (IVUS) function for generating a blood vessel tomographic image by transmitting and receiving ultrasound via a catheter 201, and an optical coherence tomography (OCT) function for generating a blood vessel tomographic image by transmitting and receiving light. The catheter 201 is a medical instrument inserted into a blood vessel of a patient, and an ultrasound transmitter and receiver for transmitting and receiving ultrasound and an optical transmitter and receiver that transmits and receives light are provided at a distal end of the catheter 201. The image diagnosis apparatus 2 generates and displays an IVUS image and an OCT image based on the ultrasound and light signals received by the catheter 201.

Note that, in the present embodiment, the image diagnosis apparatus 2 is described as an apparatus having both IVUS and OCT functions, but the image diagnosis apparatus 2 may be an apparatus having only one function. The blood vessel tomographic image may be acquired by a method other than IVUS or OCT.

The detection model 50 generated by the server 1 is installed in the image diagnosis apparatus 2, and the image diagnosis apparatus 2 inputs the blood vessel tomographic image acquired using the catheter 201 into the detection model 50, and detects a detection target region and a region different from the detection target region. As described later, the image diagnosis apparatus 2 specifies the detection target region based on the region different from the detection target region, and compares the specified detection target region with the detection target region detected by the detection model 50 to determine a boundary of the detection target region.

Note that, in the present embodiment, it is assumed that the image diagnosis apparatus 2 detects the detection target region using the detection model 50, but the server 1 in the cloud may execute processing using the detection model 50. For example, a general-purpose computer connected to the image diagnosis apparatus 2 may perform the processing. In this manner, a processing entity that executes a series of processing steps is not particularly limited.

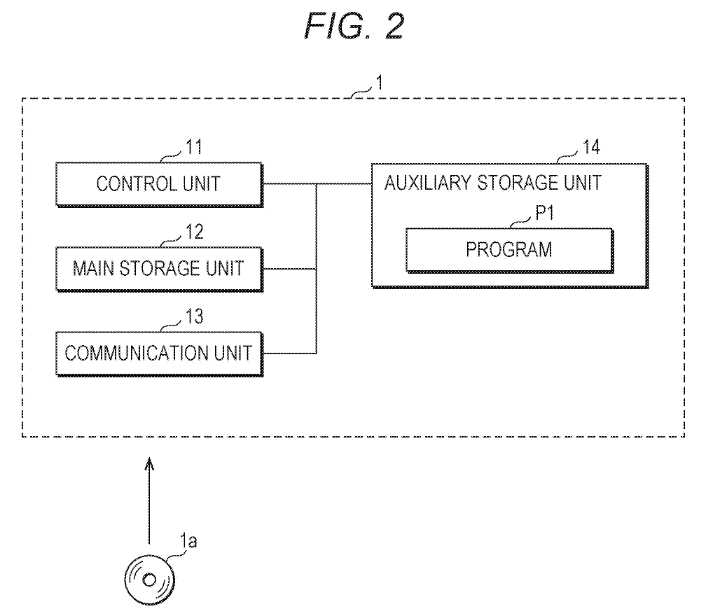

FIG. 2 is a block diagram illustrating a configuration example of the server 1. The server 1 includes a control unit 11, a main storage unit 12, a communication unit 13, and an auxiliary storage unit 14.

The control unit 11 includes one or more processors such as a central processing unit (CPU), a microprocessor unit (MPU), and a graphics processing unit (GPU), and executes various types of information processing, control processing and the like by reading and executing a program P1 stored in the auxiliary storage unit 14. The main storage unit 12 is a temporary storage area such as a static random access memory (SRAM) or a dynamic random access memory (DRAM), and temporarily stores data necessary for the control unit 11 to execute processing. The communication unit 13 is a communication module for handling communications and transmits and receives information to and from the outside. The auxiliary storage unit 14 is a non-volatile storage area such as a high-capacity memory and a hard disk, and stores the program P1 (program product) necessary for the control unit 11 to perform processing and other data.

Note that the auxiliary storage unit 14 may be an external storage device connected to the server 1. The server 1 may be a multi-computer system that includes a plurality of computers or may be a virtual machine constructed by software.

In the present embodiment, the configuration of the server 1 is not limited to the above configuration and may include an input unit that receives an user input, a display unit that displays an image and the like, for example. The server 1 may include a reading unit that reads from a portable storage medium 1a such as a compact disc (CD-ROM) and a digital versatile disc (DVD-ROM), and may read the program P1 from the portable storage medium 1a and execute the program.

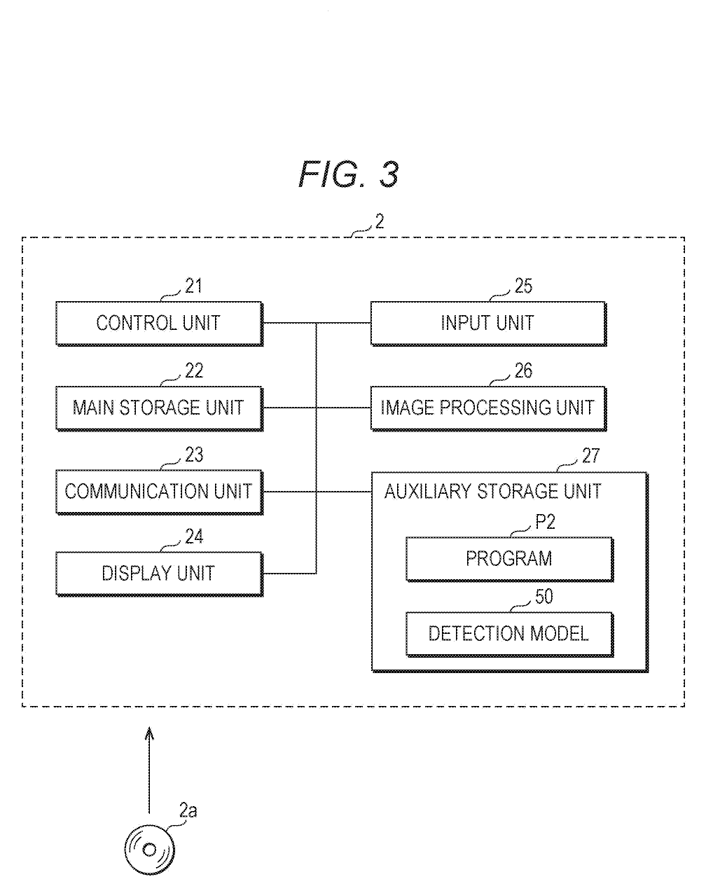

FIG. 3 is a block diagram illustrating a configuration example of the image diagnosis apparatus 2. The image diagnosis apparatus 2 includes a control unit 21, a main storage unit 22, a communication unit 23, a display unit 24, an input unit 25, an image processing unit 26, and an auxiliary storage unit 27.

The control unit 21 includes one or more processors such as a CPU, and executes various types of information processing, control processing and the like by reading and executing a program P2 stored in the auxiliary storage unit 27. The main storage unit 22 is a temporary storage area such as a RAM, and temporarily stores data necessary for the control unit 21 to execute processing. The communication unit 23 is a communication module for handling communications, and transmits and receives information to and from the outside. The display unit 24 is a display screen such as a liquid crystal display, and displays an image. The input unit 25 is an operation interface such as a keyboard and a mouse, and receives an operation input from a user. The image processing unit 26 is an image processing module that processes signals transmitted and received via the catheter 201 and generates a blood vessel tomographic image.

The auxiliary storage unit 27 is a non-volatile storage area such as a hard disk and a high-capacity memory, and stores the program P2 (program product) necessary for the control unit 21 to perform processing and other data. The auxiliary storage unit 27 stores the detection model 50. The detection model 50 is a machine learning model trained using predetermined training data, and is a trained model that detects a detection target region and the like using a blood vessel tomographic image acquired by the image diagnosis apparatus 2 as an input.

Note that the image diagnosis apparatus 2 may include a reading unit that reads from a portable storage medium 2a such as a CD-ROM, and may read the program P2 from the portable storage medium 2a and then execute the program.

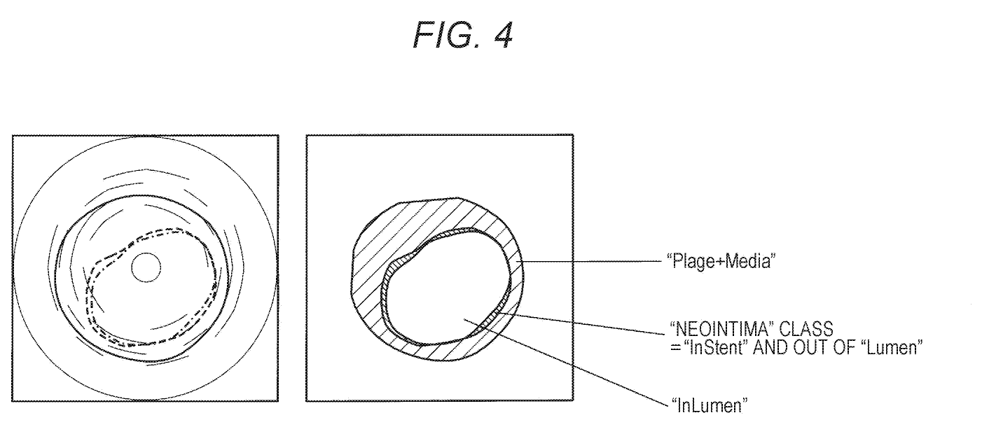

FIG. 4 is a diagram illustrating a processing example of detecting a detection target region from a blood vessel tomographic image. FIG. 4 illustrates an example of detecting a lumen of a blood vessel, a stent, an external elastic membrane (EEM) and the like from a tomographic image obtained by imaging a cross section intersecting with (orthogonal to) an axial direction of the blood vessel. With reference to FIG. 4, a disadvantage when detecting various target objects from a blood vessel tomographic image using a machine learning model will be described below.

Note that, in the present embodiment, the term “blood vessel tomographic image” basically refers to a tomographic image (an image obtained by capturing the cross section intersecting with the axial direction of the blood vessel), but the present embodiment is not limited thereto, and the detection target may be detected from a longitudinal tomographic image of the blood vessel (an image obtained by imaging a cross section in the axial direction of the blood vessel).

When detecting a desired detection target object from the blood vessel tomographic image, it is conceivable to perform semantic segmentation processing using, for example, a machine learning model. Semantic segmentation is a method of identifying a target object on a pixel-by-pixel basis from an image, and is executed using, for example, a convolutional neural network (CNN) such as U-Net.

It is possible to detect a large number of target objects from one image by training a machine learning model for semantic segmentation to learn features of various target objects. For example, as illustrated on the left side of FIG. 4, the lumen of the blood vessel, the external elastic membrane, and a boundary of the stent placed in the blood vessel can be detected from the blood vessel tomographic image, and a region of each target object can be identified on a pixel-by-pixel basis as indicated by hatching in the center of FIG. 4.

However, there is a disadvantage that, as a single model is trained to learn a large number of detection targets, a problem to be solved by the model becomes more complex and accuracy decreases. It is necessary to prepare training data for each segmented region, and there is a disadvantage of increased cost.

In order to address the above-described disadvantage, it is conceivable to create a dedicated model for each detection target and divide a detection task into a plurality of subtasks. However, there arises a disadvantage that a computational load increases due to an increase in the number of models, and a processing speed of the computer is reduced. The required storage capacity of the computer increases due to the increase in the number of models, and a hardware cost increases.

Therefore, in the present embodiment, a mode in which detection is performed using a single model while improving detection accuracy by dividing the detection task into a plurality of subtasks will be described.

FIG. 5 is a diagram illustrating an outline of the first embodiment. FIG. 5 illustrates a state in which the same detection model 50 detects a detection target region (“inLumen”) indicating a main trunk lumen of the blood vessel and a region (“inLumen_background”) different from the detection target region, and the boundary of the detection target region is finally determined by combining detection results of both regions. With reference to FIG. 5, the outline of the present embodiment will be described below.

The detection model 50 is a machine learning model that has learned the predetermined training data, and is a model that detects, for one or a plurality of detection targets, the detection target region indicating the detection target and the region different from the detection target region in a case where the blood vessel tomographic image is input. For example, the detection model 50 is a model (CNN) related to semantic segmentation such as U-net, and detects the detection target region and the region different from the detection target region on a pixel-by-pixel basis.

Note that the detection model 50 may be a neural network other than the CNN. The detection model 50 may be, for example, a machine learning model other than a neural network, such as a decision tree or a support vector machine (SVM).

In the present embodiment, the blood vessel tomographic image generated (constructed) from data of a reflected signal of the ultrasound is used as the input to the detection model 50, but the present embodiment is not limited thereto, and raw data of the reflected signal serving as the basis of the blood vessel tomographic image may be used as the input to the detection model 50. That is, it is sufficient that the detection model 50 can receive data indicating the blood vessel tomographic image as the input, and the data is not limited to image data.

Although the image diagnosis apparatus 2 according to the present embodiment captures the IVUS image (ultrasound tomographic image) and the OCT image (optical coherence tomographic image) as described above, separate detection models 50 may be constructed according to the respective images, or the detection target region may be detected from the respective images using a common detection model 50.

The detection target region includes at least one of a region indicating the inside of the stent, a region indicating the main trunk lumen of the blood vessel, a region indicating the inside of the external elastic membrane of the main trunk of the blood vessel, a region indicating the inside of an outer membrane of the main trunk of the blood vessel, a region indicating a side branch lumen of the blood vessel, a region indicating the inside of the external elastic membrane of the side branch of the blood vessel, a region indicating the lumen of the main trunk and the side branch of the blood vessel, a region indicating the inside of the external elastic membrane of the main trunk and the side branch of the blood vessel, a plaque region, a thrombus region, a haematoma region, a calcified region, a dissection region, a crack region, an extravascular tissue region, and a device region. Note that the “device” refers to a device such as a guide wire inserted into the blood vessel of the patient.

Note that the above description is an example of the detection target region, and another detection target may be detected. The detection model 50 according to the present embodiment detects, for the plurality of detection targets, the detection target regions indicating the detection targets. In FIG. 5, as an example, a case of detecting the main trunk lumen of the blood vessel will be described.

The region different from the detection target region refers to a region of a target object different from the detection target for which the boundary is to be finally determined. In the present embodiment, in order to finally determine the boundary of the main trunk lumen of the blood vessel, a region excluding a region (detection target region) indicating the main trunk lumen of the blood vessel is detected as the region different from the detection target region.

The server 1 generates the detection model 50 by learning the training data for generating the detection model 50. The training data is data in which a label indicating a correct position of the detection target region (region indicating the main trunk lumen of the blood vessel) is assigned to a blood vessel tomographic image for training.

The server 1 reverses the detection target region indicated by the label assigned to the blood vessel tomographic image for training, thereby specifying the region excluding the detection target region (region indicating the outside of the main trunk lumen of the blood vessel). The server 1 assigns a label indicating the position of the specified region to the blood vessel tomographic image for training.

The server 1 generates the trained detection model 50 so as to detect the detection target region and the region excluding the detection target region in a case where the blood vessel tomographic image is input on the basis of the training data to which the label is newly assigned. The server 1 detects the detection target region and the region excluding the detection target region by inputting the blood vessel tomographic image for training to the detection model 50. Specifically, by inputting the blood vessel tomographic image for training to the detection model 50, the server 1 outputs label data (label image) indicating whether each pixel belongs to a target region in binary for each of the detection target region and the region excluding the detection target region. The server 1 compares each detected region with a region indicated by a correct label, and adjusts parameters such as weights between neurons so that both regions coincide with each other. The server 1 sequentially inputs each blood vessel tomographic image for training to the detection model 50 to perform learning, and finally generates the detection model 50 with optimized parameters.

The image diagnosis apparatus 2 detects the detection target region using the detection model 50 generated as described above. First, as illustrated in FIG. 5, the image diagnosis apparatus 2 inputs the blood vessel tomographic image to the detection model 50 to detect the detection target region (region indicating the main trunk lumen of the blood vessel) and the region excluding the detection target region (region indicating the outside of the main trunk lumen of the blood vessel). Specifically, by inputting the blood vessel tomographic image to the detection model 50, the image diagnosis apparatus 2 outputs the label data (label image) indicating whether each pixel belongs to a target region in binary for each of the detection target region and the region excluding the detection target region.

Next, the image diagnosis apparatus 2 specifies the detection target region by reversing the region excluding the detection target region. The image diagnosis apparatus 2 compares the detection target region specified by reversing the region excluding the detection target region with the detection target region detected by the detection model 50, and determines the boundary of the detection target region. For example, the image diagnosis apparatus 2 determines that a region (pixel) belonging to either of them is the detection target region (OR operation) and determines the boundary. Alternatively, the image diagnosis apparatus 2 may determine that a region (pixel) belonging to both regions is the detection target region (AND operation) and determine the boundary.

The image diagnosis apparatus 2 displays the detection result of the detection target region. For example, similarly to FIG. 4, the image diagnosis apparatus 2 displays the blood vessel tomographic image in which a boundary line is superimposed on the detection target region (the main trunk lumen of the blood vessel). The image diagnosis apparatus 2 calculates a lumen diameter and the like of the blood vessel on the basis of the boundary line of the lumen, and displays the same together with the blood vessel tomographic image.

Although the main trunk lumen of the blood vessel has been described above as an example of the detection target, the image diagnosis apparatus 2 similarly detects the detection target region by the detection model 50 for other detection targets. That is, the image diagnosis apparatus 2 detects the region indicating the inside of the stent, the region indicating the inside of the external elastic membrane of the main trunk of the blood vessel and the like as the detection target region by the detection model 50, detects the region (reversed region) excluding the detection target region, and determines the boundary of the detection target region by combining both regions. The image diagnosis apparatus 2 also superimposes and displays the boundary line on the blood vessel tomographic image for these detection targets, and calculates and displays the diameter (EEM diameter) of the external elastic membrane.

In this manner, the image diagnosis apparatus 2 detects the detection target region and the region different from the detection target region (the region excluding the detection target region) by the detection model 50, and determines the boundary of the detection target region on the basis of the detection target region and the region different from the detection target region. This makes it possible to suppress an increase in computational cost while improving the detection accuracy.

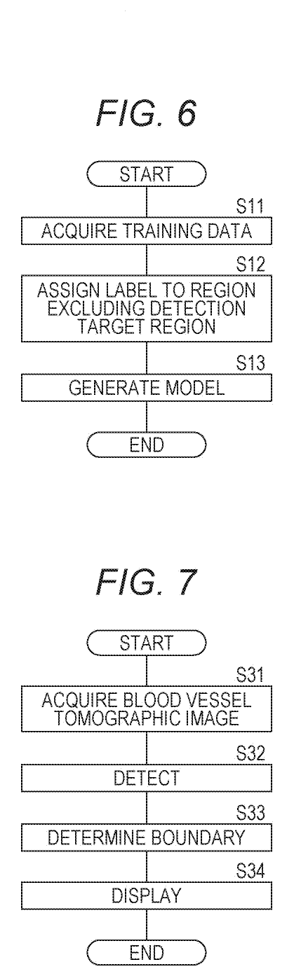

FIG. 6 is a flowchart illustrating a procedure of generation processing of the detection model 50. Processing contents when generating the detection model 50 by machine learning will be described with reference to FIG. 6.

The control unit 11 of the server 1 acquires the training data for generating the detection model 50 (S11). The training data is data in which the label indicating the correct position of the detection target region is assigned to the blood vessel tomographic image for training. The blood vessel tomographic image is, for example, the tomographic image obtained by imaging the cross section intersecting with (orthogonal to) the axial direction of the blood vessel. The detection target region is, for example, the region indicating the main trunk lumen of the blood vessel, but is not limited thereto.

The control unit 11 reverses the detection target region indicated by the training data to assign a label indicating the position of the region excluding the detection target region to the blood vessel tomographic image for training (S12).

The control unit 11 generates the trained detection model 50 so as to detect the detection target region and the region excluding the detection target region in a case where the blood vessel tomographic image is input on the basis of the training data (S13). For example, the control unit 11 generates the neural network such as the CNN as the detection model 50. The control unit 11 detects the detection target region and the region excluding the detection target region by inputting the blood vessel tomographic image for training to the detection model 50. The control unit 11 generates the detection model 50 by comparing each detected region with a correct region and optimizing the parameters such as weights between neurons so that both regions coincide with each other. The control unit 11 ends a series of processing.

FIG. 7 is a flowchart illustrating detection processing of the detection target region. Processing contents when detecting the detection target region using the detection model 50 and determining the boundary thereof will be described with reference to FIG. 7.

The control unit 21 of the image diagnosis apparatus 2 acquires the blood vessel tomographic image obtained by imaging the blood vessel cross section of the patient (S31). The control unit 21 inputs the acquired blood vessel tomographic image to the detection model 50 to detect the detection target region (such as the region indicating the main trunk lumen of the blood vessel) and the region excluding the detection target region (the region obtained by reversing the detection target region) (S32).

The control unit 21 determines the boundary of the detection target region by combining the detection target region and the region excluding the detection target region detected at S32 (S33). Specifically, the control unit 21 specifies the detection target region by reversing the region excluding the detection target region. The control unit 21 determines the boundary of the detection target region by performing the OR operation or AND operation between the specified detection target region and the detection target region detected by the detection model 50.

The control unit 21 displays the blood vessel tomographic image in which the boundary line is superimposed on the detection target region (S34), and ends a series of processing.

As described above, according to the first embodiment, the boundary of the detection target region in the blood vessel tomographic image can suitably be determined.

### Second Embodiment

In a second embodiment, a mode of detecting a side branch of a blood vessel from a blood vessel tomographic image will be described. Note that the same reference numerals are given to the same contents as those of the first embodiment, and the description thereof will be omitted.

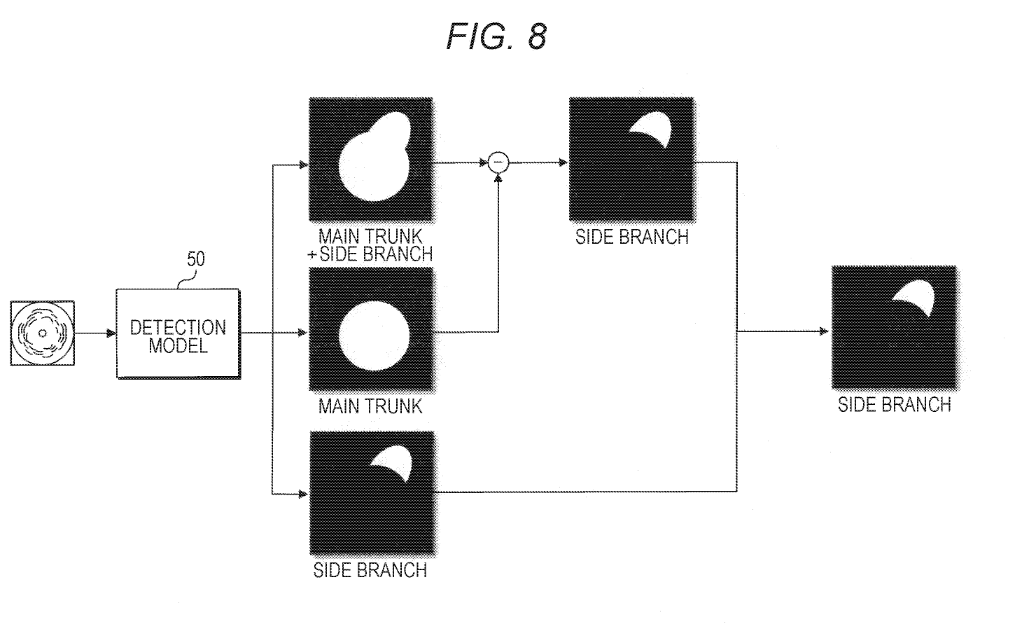

FIG. 8 is a diagram illustrating an outline of the second embodiment. On the basis of the second embodiment, an outline of the present embodiment will be described.

In the present embodiment, a detection model 50 detects a side branch of a blood vessel in addition to a main trunk lumen of the blood vessel, an external elastic membrane of the main trunk and the like. Specifically, as illustrated in FIG. 8, the detection model 50 detects a region indicating the side branch of the blood vessel as a detection target region, and detects a region indicating the main trunk of the blood vessel and a region indicating the main trunk and side branch of the blood vessel as regions different from the detection target region.

Note that the “region indicating the side branch of the blood vessel” may be a region indicating a side branch lumen of the blood vessel or a region indicating the inside of an external elastic membrane of the side branch. Similarly, the “region indicating the main trunk of the blood vessel” and the “region indicating the main trunk and side branch of the blood vessel” may be regions indicating the lumen or may be regions indicating the inside of the external elastic membrane. In the present embodiment, both the lumen and the external elastic membrane are detected. FIG. 8 illustrates a case where the lumen is detected as an example.

A server 1 generates the detection model 50 using training data in which labels indicating positions of a correct region indicating the side branch of the blood vessel, the region indicating the main trunk of the blood vessel, and the region indicating the main trunk and side branch of the blood vessel are assigned to a blood vessel tomographic image for training.

An image diagnosis apparatus 2 detects the region indicating the side branch of the blood vessel using the detection model 50 generated as described above. First, the image diagnosis apparatus 2 inputs the blood vessel tomographic image to the detection model 50 to detect the region indicating the side branch of the blood vessel, the region indicating the main trunk of the blood vessel, and the region indicating the main trunk and side branch of the blood vessel. Next, the image diagnosis apparatus 2 specifies the region indicating the side branch of the blood vessel by excluding the region indicating the main trunk of the blood vessel from the detected region indicating the main trunk and side branch of the blood vessel.

The image diagnosis apparatus 2 determines a boundary of the region indicating the side branch of the blood vessel by combining the specified region indicating the side branch of the blood vessel and the region indicating the side branch of the blood vessel detected by the detection model 50. That is, the image diagnosis apparatus 2 determines the boundary of the region indicating the side branch of the blood vessel by performing an OR operation or an AND operation on both regions.

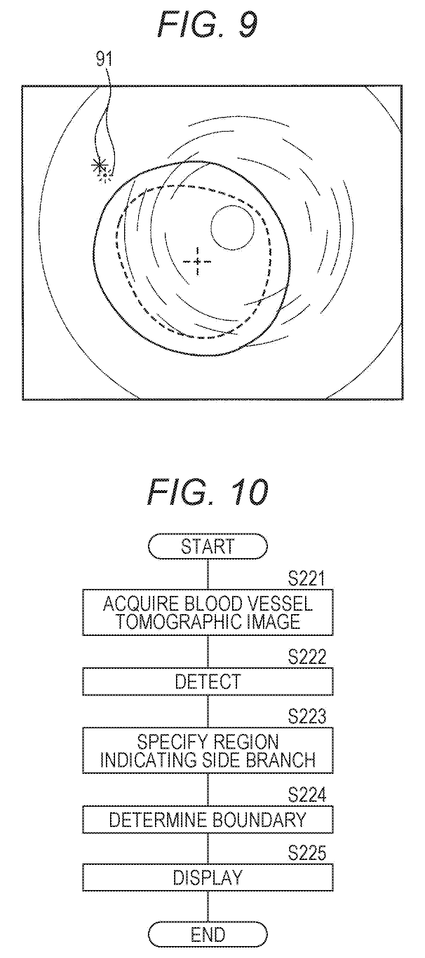

FIG. 9 is a diagram illustrating a display example of a detection result of the side branch of the blood vessel. Similarly to the first embodiment, the image diagnosis apparatus 2 displays a detection result of the detection target region (the region indicating the side branch of the blood vessel in the present embodiment). Here, the image diagnosis apparatus 2 superimposes and displays a marker 91 indicating a representative position of the side branch on the blood vessel tomographic image (tomographic image) on the basis of the boundary of the region indicating the side branch of the blood vessel determined above. For example, the image diagnosis apparatus 2 specifies the center of gravity of the region indicating the side branch of the blood vessel as a representative position of the side branch, and displays a marker 91 at the specified representative position.

Note that two markers 91 are displayed in FIG. 9, which represent the representative position of the side branch with reference to the lumen of the blood vessel and the representative position of the side branch with reference to the external elastic membrane of the blood vessel. The image diagnosis apparatus 2 detects the region indicating the lumen of the main trunk of the blood vessel and the region indicating the inside of the external elastic membrane of the main trunk of the blood vessel by the detection model 50, superimposes and displays a boundary line indicating the lumen of the main trunk of the blood vessel and a boundary line indicating the external elastic membrane of the main trunk of the blood vessel on the blood vessel tomographic image, and superimposes and displays the marker 91 indicating the representative position of the side branch with reference to the lumen of the blood vessel and the marker 91 indicating the representative position of the side branch with reference to the external elastic membrane of the blood vessel.

FIG. 10 is a flowchart illustrating a procedure of detection processing of the detection target region according to the second embodiment.

A control unit 21 of the image diagnosis apparatus 2 acquires the blood vessel tomographic image (S221). The control unit 21 inputs the acquired blood vessel tomographic image to the detection model 50 to detect the region indicating the side branch of the blood vessel (detection target region), the region indicating the main trunk of the blood vessel, and the region indicating the main trunk and side branch of the blood vessel (region different from the detection target region) (S222).

The control unit 21 specifies the region indicating the side branch of the blood vessel by excluding the region indicating the main trunk of the blood vessel from the detected region indicating the main trunk and lumen of the blood vessel (S223). The control unit 21 determines the boundary of the region indicating the side branch of the blood vessel by combining the specified region indicating the side branch of the blood vessel and the region indicating the side branch of the blood vessel detected at S222 (S224). On the basis of the determined boundary of the region indicating the side branch of the blood vessel, the control unit 21 displays the blood vessel tomographic image (tomographic image) on which the marker indicating the representative position of the side branch is superimposed (S225), and ends a series of processing.

As described above, according to the second embodiment, the boundary of the region indicating the side branch of the blood vessel can be suitably determined.

### Third Embodiment

In a third embodiment, a mode of detecting a calcified region from a blood vessel tomographic image will be described.

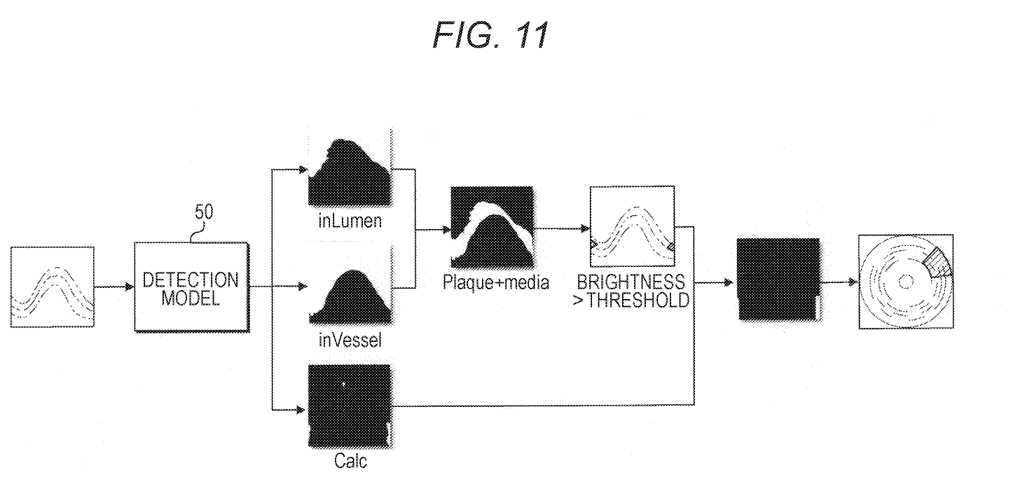

FIG. 11 is a diagram illustrating an outline of the third embodiment. With reference to FIG. 11, the outline of the present embodiment will be described.

A detection model 50 according to the present embodiment detects a calcified region in which a vascular tissue is calcified, in addition to a main trunk lumen of a blood vessel, an external elastic membrane of the main trunk and the like. Specifically, as illustrated in FIG. 11, the detection model 50 detects the calcified region as a detection target region, and detects a region indicating the inside of the external elastic membrane (of the main trunk) of the blood vessel and the region indicating the (main trunk) lumen of the blood vessel as regions different from the detection target region.

A server 1 generates the detection model 50 using training data in which labels indicating a position of a correct calcified region, a position of the region indicating the inside of the external elastic membrane of the blood vessel, and a position of the region indicating the lumen of the blood vessel are assigned to a blood vessel tomographic image for training. Note that, in the present embodiment, not an orthogonal coordinate image but a polar coordinate image (an image in which one axis represents a distance from a distal end of a catheter 201 and the other axis represents a rotation angle) is used as the blood vessel tomographic image input to the detection model 50.

An image diagnosis apparatus 2 detects the calcified region using the detection model 50 generated as described above. First, the image diagnosis apparatus 2 inputs the blood vessel tomographic image to the detection model 50 to detect a calcified region (“Calc”), a region indicating the inside of the external elastic membrane of the blood vessel (“inVessel”), and a region indicating the lumen of the blood vessel (“inLumen”).

Next, the image diagnosis apparatus 2 specifies a region indicating a vessel wall (“Plaque+media”) on the basis of the detected region indicating the inside of the external elastic membrane of the blood vessel and the region indicating the lumen of the blood vessel. The image diagnosis apparatus 2 detects, as the calcified region, a high brightness region having brightness equal to or higher than a threshold in the specified region indicating the vessel wall. Specifically, the image diagnosis apparatus 2 detects, as the calcified region, a portion (calcified surface) the brightness of which is equal to or higher than the threshold and a region (acoustic shadow) behind (below in FIG. 11) the portion as seen from the distal end of the catheter 201. Note that the threshold as a reference for detecting the calcified region may be determined according to a brightness value of an outer membrane of the blood vessel.

The image diagnosis apparatus 2 determines a boundary of the calcified region by combining the calcified region (high brightness region) detected above and the calcified region detected by the detection model 50. Specifically, the image diagnosis apparatus 2 calculates a degree of coincidence (for example, a Jaccard coefficient) between both regions, determines that a region where the degree of coincidence is equal to or higher than a specified value is the calcified region, and determines the boundary.

Note that the above-described method of detecting the calcified region is processing mainly applied to an IVUS image, and the calcified region can be usually observed as a region having a thickness in a depth direction from the image center in an OCT image, so that correction by brightness (detection of the high brightness region and determination of the boundary) is not performed in the OCT image, and only detection of the calcified region by the detection model 50 is performed.

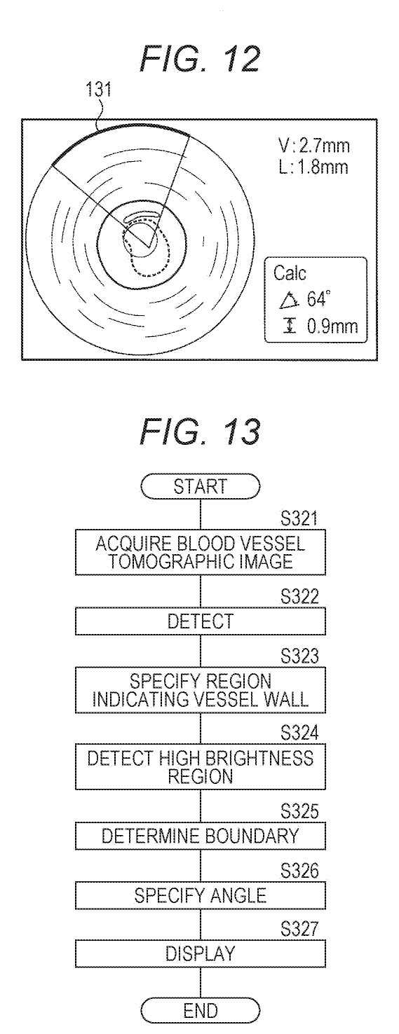

FIG. 12 is a diagram illustrating a display example of a detection result of the calcified region. Similarly to the first and second embodiments, the image diagnosis apparatus 2 displays the detection result of the detection target region (calcified region). In the present embodiment, the image diagnosis apparatus 2 specifies an angle occupied by the calcified region to be detected in a circumferential direction of the blood vessel, and displays a blood vessel tomographic image (tomographic image) indicating the angle.

Specifically, as illustrated in FIG. 12, the image diagnosis apparatus 2 superimposes and displays an arc 131 on the tomographic image according to an angular range of the calcified region as seen from the center of gravity of the lumen of the blood vessel. The image diagnosis apparatus 2 displays the angle of the calcified region and a thickness of the calcified region in a lower right portion of the screen. Note that, since the thickness of the calcified region cannot be identified from the IVUS image, this is identified from the detection result of the calcified region by the OCT image.

FIG. 13 is a flowchart illustrating detection processing of the detection target region according to the third embodiment.

A control unit 21 of the image diagnosis apparatus 2 acquires the blood vessel tomographic image (S321). The control unit 21 inputs the acquired blood vessel tomographic image to the detection model 50 to detect the calcified region (detection target region), the region indicating the inside of the external elastic membrane of the blood vessel, and the region indicating the lumen of the blood vessel (region different from the detection target region) (S322).

The control unit 21 specifies a region indicating a vessel wall on the basis of the detected region indicating the inside of the external elastic membrane of the blood vessel and the region indicating the lumen of the blood vessel (S323). The control unit 21 detects a high brightness region having brightness equal to or higher than a threshold in the specified region indicating the vessel wall (S324). The control unit 21 determines the boundary of the calcified region by combining the high brightness region and the calcified region detected by the detection model 50 (S325).

The control unit 21 specifies the angle occupied by the calcified region in the circumferential direction of the blood vessel on the basis of the determined boundary of the calcified region (S326). The control unit 21 displays the blood vessel tomographic image (tomographic image) indicating the specified angle (S327), and ends a series of processing.

As described above, according to the third embodiment, the boundary of the calcified region can be suitably determined.

### Modification

In the third embodiment, the mode of detecting the calcified region from the blood vessel tomographic image has been described. In this modification, a mode of determining a feature of calcification on the basis of the position of the detected calcified region within a vessel wall will be described.

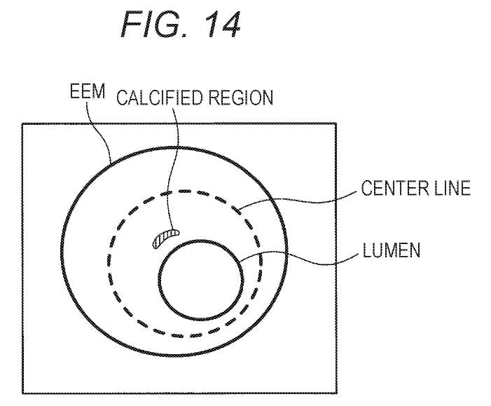

FIG. 14 is a diagram illustrating an outline of the modification. In FIG. 14, a solid line indicates a lumen and an external elastic membrane of a blood vessel, a broken line indicates a centerline between the lumen and the external elastic membrane of the blood vessel, and a hatched portion indicates the calcified region. With reference to FIG. 14, an outline of this modification will be described.

As described in the third embodiment, the image diagnosis apparatus 2 specifies the region indicating the vessel wall using the detection model 50, specifies a region having brightness equal to or higher than the threshold within the region indicating the vessel wall as the calcified region, and compares the region with the calcified region detected by the detection model 50 to determine the boundary of the calcified region. In this modification, the image diagnosis apparatus 2 determines a feature of calcification such as whether the calcification is is shallow, deep, or superficial on the basis of the position of the calcified region, the boundary of which has been determined, with respect to the region indicating the vessel wall.

Specifically, the image diagnosis apparatus 2 specifies the centerline between the lumen and the external elastic membrane of the blood vessel based on the region indicating the vessel wall. The image diagnosis apparatus 2 determines whether the calcified region is located inside the centerline and whether the calcified region is in contact with the lumen of the blood vessel. In a case where it is determined that the calcified region is located outside the centerline, the image diagnosis apparatus 2 determines that the calcification is deep. In a case where it is determined that the calcified region is located inside the centerline and not in contact with the lumen, the image diagnosis apparatus 2 determines that the calcification is shallow. In a case where it is determined that the calcified region is in contact with the lumen, the image diagnosis apparatus 2 determines that the calcification is superficial.

In this manner, the image diagnosis apparatus 2 determines the feature of calcification according to the position of the calcified region in the region indicating the vessel wall.

Since this modification is similar to the third embodiment except for the above-described points, detailed description of the flowchart and the like is omitted herein.

### Fourth Embodiment

In a fourth embodiment, a mode of detecting an attenuated plaque region from a blood vessel tomographic image will be described.

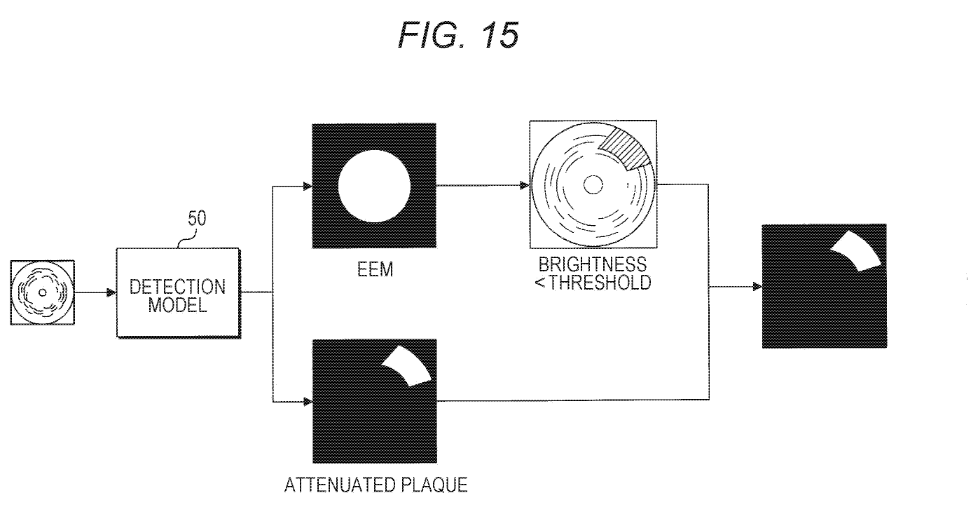

FIG. 15 is a diagram illustrating an outline of the fourth embodiment. With reference to FIG. 15, the outline of the present embodiment will be described.

A detection model 50 according to the present embodiment detects an attenuated plaque region in which a ultrasound signal is attenuated in an IVUS image, in addition to an external elastic membrane of the main trunk of a blood vessel and the like. Specifically, as illustrated in FIG. 15, in a case where the blood vessel tomographic image is input, the detection model 50 detects the attenuated plaque region as a detection target region, and detects a region indicating the inside of the external elastic membrane of the blood vessel as a region different from the detection target region.

A server 1 generates the detection model 50 using training data in which labels indicating a position of a correct attenuated plaque region, and a position of the region indicating the inside of the external elastic membrane of the blood vessel are assigned to a blood vessel tomographic image for training.

The image diagnosis apparatus 2 detects the attenuated plaque region from the blood vessel tomographic image using the detection model 50 generated as described above. First, the image diagnosis apparatus 2 inputs the blood vessel tomographic image to the detection model 50 to detect the attenuated plaque region and the region indicating the inside of the external elastic membrane of the blood vessel.

Next, the image diagnosis apparatus 2 detects, as the attenuated plaque region, a low brightness region having brightness equal to or lower than a threshold outside the region indicating the inside of the external elastic membrane of the blood vessel. For example, the image diagnosis apparatus 2 sets the threshold based on the brightness of a region outside the external elastic membrane, and detects a region in which the brightness is equal to or lower than the threshold.

The image diagnosis apparatus 2 determines a boundary of the attenuated plaque region by combining the attenuated plaque region (low brightness region) detected above and the attenuated plaque region detected by the detection model 50. The image diagnosis apparatus 2 specifies a common region by performing an OR operation or an AND operation on both regions, and determines the boundary.

Similarly to the first to third embodiments, the image diagnosis apparatus 2 displays a detection result of a detection target region (attenuated plaque region). In the present embodiment, as in the third embodiment, the image diagnosis apparatus 2 specifies an angle occupied by the attenuated plaque region in a circumferential direction of the blood vessel (the angle in a case where the center of gravity of the lumen of the blood vessel is set as the center) on the basis of the boundary of the attenuated plaque region determined above, and displays a blood vessel tomographic image (tomographic image) indicating the specified angle as an arc (refer to FIG. 12).

Note that the image diagnosis apparatus 2 may simultaneously detect a calcified region by the method described in the third embodiment together with the attenuated plaque region. Here, in a case where the angle occupied by the attenuated plaque region and the calcified region in the circumferential direction of the blood vessel is displayed as the arc 131 (refer to FIG. 12), the image diagnosis apparatus 2 suitably displays the arc 131 indicating the angle of each region in a different display mode (for example, different display colors).

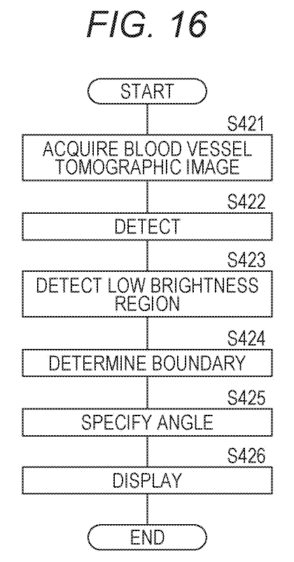

FIG. 16 is a flowchart illustrating detection processing of the detection target region according to the fourth embodiment.

A control unit 21 of the image diagnosis apparatus 2 acquires the blood vessel tomographic image (S421). The control unit 21 inputs the acquired blood vessel tomographic image to the detection model 50 to detect the attenuated plaque region and the region indicating the inside of the external elastic membrane of the blood vessel (S422).

The control unit 21 detects a low brightness region in which brightness is equal to or lower than a threshold outside the detected region indicating the inside of the external elastic membrane of the blood vessel (S423). The control unit 21 determines a boundary of the attenuated plaque region by combining the low brightness region and the attenuated plaque region detected by the detection model 50 (S424).

The control unit 21 specifies the angle occupied by the attenuated plaque region in the circumferential direction of the blood vessel on the basis of the determined boundary of the attenuated plaque region (S425). The control unit 21 displays the blood vessel tomographic image (tomographic image) indicating the specified angle (S426), and ends a series of processing.

As described above, according to the fourth embodiment, the boundary of the attenuated plaque region can be suitably determined.

### Fifth Embodiment

In the first to fourth embodiments, the mode of detecting the lumen, the side branch, the calcified region and the like of the blood vessel has been described. In a fifth embodiment, a mode of presenting (displaying) a detection result of each region will be described.

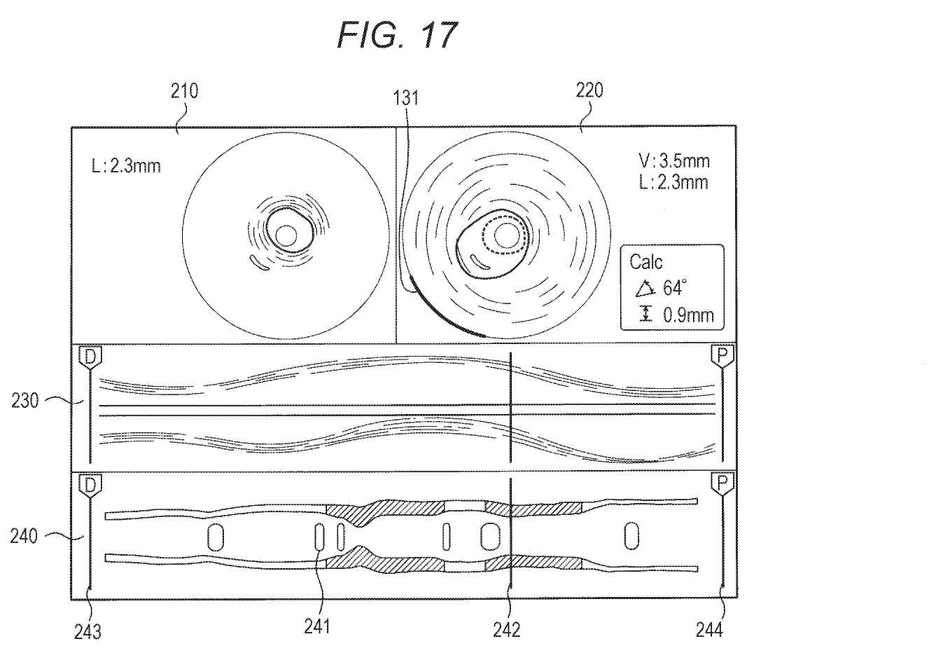

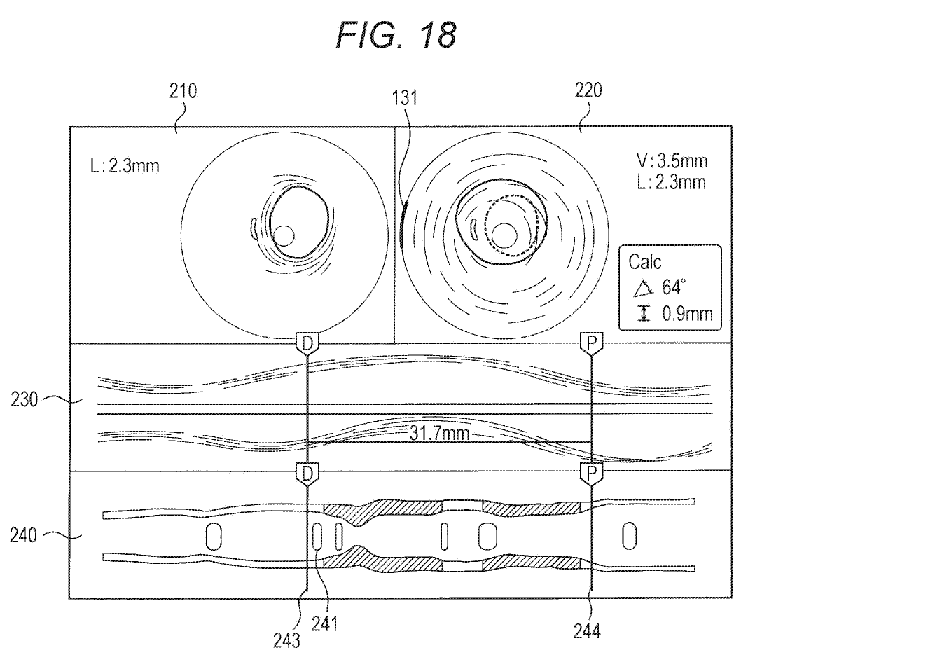

FIGS. 17 and 18 are diagrams illustrating an outline of the fifth embodiment. FIGS. 17 and 18 illustrate an example analysis screen of a blood vessel tomographic image displayed by an image diagnosis apparatus 2 on the basis of the detection result of each detection target region by a detection model 50. With reference to FIGS. 17 and 18, the outline of the present embodiment will be described.

The image diagnosis apparatus 2 captures a blood vessel tomographic image (tomographic image) at each position in an axial direction of the blood vessel in accordance with a pullback operation of a catheter 201. The image diagnosis apparatus 2 detects the detection target region of each type from the blood vessel tomographic image at each position by the method described in the first to fourth embodiments. That is, the image diagnosis apparatus 2 detects a region indicating a lumen of a blood vessel, a region indicating the inside of an external elastic membrane, a region indicating a side branch, a plaque region, a calcified region and the like, and determines a boundary thereof.

On an analysis screen illustrated in FIGS. 17 and 18, an image for a user (medical worker) to determine a deployment position (landing zone) of a stent and the like is displayed using a detection result of the detection target region.

The analysis screen includes a first image display field 210, a second image display field 220, a third image display field 230, and a fourth image display field 240. The first image display field 210 is a display field in which an OCT image (tomographic image) is displayed. The second image display field 220 is a display field in which an IVUS image (tomographic image) is displayed. The third image display field 230 and the fourth image display field 240 are display fields in which a longitudinal tomographic image of the blood vessel is displayed.

The image diagnosis apparatus 2 generates (reconstructs) a longitudinal tomographic image from the tomographic images (for example, IVUS images) continuously captured in the axial direction of the blood vessel and displays the same in the third image display field 230. The image diagnosis apparatus 2 displays the longitudinal tomographic image in which the display color is changed or a marker 241 is displayed according to an amount of plaque and the presence or absence of a side branch in the fourth image display field 240.

Specifically, the image diagnosis apparatus 2 calculates a ratio of the plaque region to a cross sectional area of the blood vessel at each position in the axial direction on the basis of an area of the plaque region and an area of the region indicating the lumen of the blood vessel in respective tomographic images continuously captured in the axial direction of the blood vessel. The ratio is calculated by, for example, (area of plaque region)/(area of plaque region + area of region indicating lumen of blood vessel). The image diagnosis apparatus 2 displays the vessel wall at each position in the axial direction on the longitudinal tomographic image in different display modes according to the calculated ratio. For example, the image diagnosis apparatus 2 displays a vessel wall portion in which a calculated ratio is equal to or higher than a threshold in a display color different from that of other portions. Note that, in FIGS. 17 and 18, a state of different display colors is illustrated by hatching for convenience.

The image diagnosis apparatus 2 displays the marker 241 at each position on the longitudinal tomographic image according to whether the region indicating the side branch of the blood vessel is detected from the respective tomographic images captured in the axial direction of the blood vessel. Note that, in a case where a cursor 242 to be described later is located on the marker 241 on the longitudinal tomographic image, a marker 91 indicating a representative position of the side branch is displayed on the tomographic image displayed in the first image display field 210 or the second image display field 220 (refer to FIG. 9).

Note that, in FIGS. 17 and 18, a vertical rod-shaped marker 241 is illustrated as the marker 241 indicating the side branch, but for example, the marker 241 may be a mark (for example, a triangle mark) indicating a frame at a representative position of each side branch.

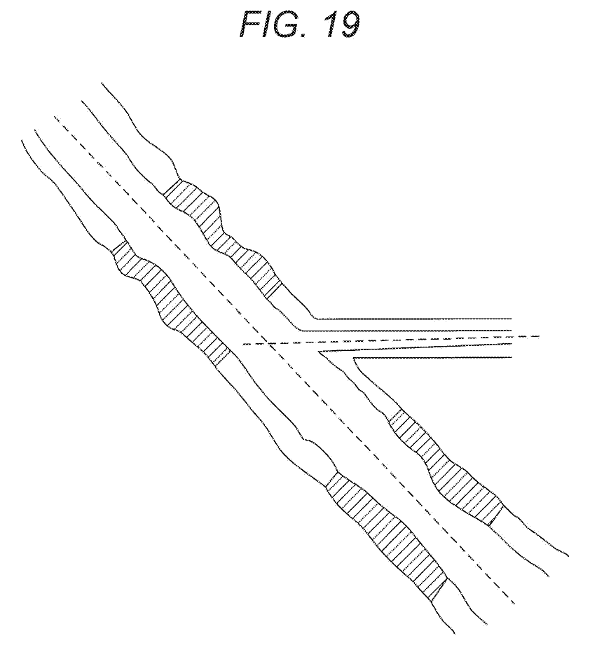

In a case of receiving an operation input for designating the marker 241, the image diagnosis apparatus 2 may generate and display a longitudinal tomographic image illustrating a longitudinal cross section along the centerline of the side branch corresponding to the designated marker 241. FIG. 19 is an explanatory view illustrating a longitudinal tomographic image along the centerline of the side branch. In a case of receiving a designation input of the marker 241, as illustrated in FIG. 19, the image diagnosis apparatus 2 displays a longitudinal tomographic image obtained by cutting the blood vessel so as to pass through the centerline of the main trunk of the blood vessel (or the centerline of the tomographic image) and the centerline of the side branch, with the centerline of the side branch as a lateral direction. As a result, the user can appropriately grasp the structure of the blood vessel.

Referring back to FIGS. 17 and 18, the description is continued. The image diagnosis apparatus 2 displays the cursor 242 on the longitudinal tomographic image displayed in the third image display field 230 and the fourth image display field 240. The cursor 242 is an object for designating the position of the tomographic image (OCT image and IVUS image) displayed in the first image display field 210 and the second image display field 220. Note that the cursor 242 is initially positioned at a position where the blood vessel is narrowed the most (the position where the diameter of the lumen of the blood vessel is the smallest).

The image diagnosis apparatus 2 receives an operation input for moving the cursor 242 in the axial direction (lateral direction) from the user. A tomographic image corresponding to the position of the cursor 242 is displayed in each of the first image display field 210 and the second image display field 220 in accordance with a moving operation of the cursor 242.

The image diagnosis apparatus 2 displays the tomographic image corresponding to the position of the cursor 242 in the first image display field 210 and the second image display field 220, and superimposes a boundary line indicating the lumen of the blood vessel, a boundary line indicating the external elastic membrane of the blood vessel and the like on the tomographic image. The image diagnosis apparatus 2 calculates a lumen diameter (L), a blood vessel diameter (diameter of the external elastic membrane, V) and the like of the blood vessel, and displays the same in association with the tomographic image.

In a case where the calcified region and attenuated plaque region are detected from the tomographic image, the image diagnosis apparatus 2 displays the arc 131 indicating the angle occupied by the region in the circumferential direction of the blood vessel in the tomographic image. The image diagnosis apparatus 2 displays the arc 131 and also displays information such as the angle and a thickness of the calcified region and the like. This makes it possible to appropriately study a method of vessel preparation.

As described above, the user operates the cursor 242 to confirm the tomographic image at each position, and is presented with the lumen diameter and the like. The user also moves reference cursors 243 and 244. In a case where positions of the reference cursors 243 and 244 are determined, the image diagnosis apparatus 2 calculates and displays a distance between the reference cursors 243 and 244 as illustrated in FIG. 18. As a result, the deployment position (landing zone) of the stent can be determined, and a stent size can be appropriately determined.

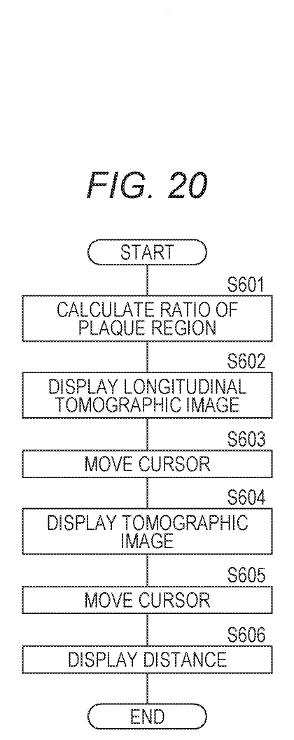

FIG. 20 is a flowchart illustrating display processing of the blood vessel image. Note that the description assumes that the image diagnosis apparatus 2 has detected the detection target region using the detection model 50 in advance, thereby detecting the lumen, side branch, plaque region and the like of the blood vessel from the blood vessel tomographic image.

The control unit 21 of the image diagnosis apparatus 2 calculates the ratio of the plaque region to the cross sectional area of the blood vessel at each position in the axial direction on the basis of the area of the plaque region and the area of the region indicating the lumen of the blood vessel in respective tomographic images continuously captured in the axial direction of the blood vessel (S601). On the basis of the respective tomographic images continuously captured in the axial direction of the blood vessel, the control unit 21 generates and displays the longitudinal tomographic image displaying the vessel wall at each position in the axial direction in a different display mode (for example, different display color) according to the ratio calculated at S601 (S602). Specifically, as described above, the control unit 21 changes the display color of the vessel wall according to the ratio of the plaque region, and generates and displays the longitudinal tomographic image in which the marker 241 is displayed at each position in the axial direction according to whether the region indicating the side branch of the blood vessel is detected from each tomographic image.

The control unit 21 receives an operation input for moving the cursor 242 displayed on the longitudinal tomographic image in the axial direction (S603). The control unit 21 displays the tomographic image corresponding to the position where the cursor 242 has moved in the first image display field 210 and the second image display field 220 (S604).

The control unit 21 receives an operation input for moving the reference cursors 243 and 244 displayed on the longitudinal tomographic image in the axial direction (S605). The control unit 21 calculates and displays the distance between the reference cursors 243 and 244 (S606), and ends a series of processing.

As described above, according to the fifth embodiment, it is possible to suitably support a user (medical worker) who performs intravascular treatment such as PCI.

It should be understood that the embodiments disclosed herein are illustrative in all respects and are not restrictive. The scope of the present disclosure is indicated not by the foregoing description but by the claims and is intended to include all changes within the meaning and scope equivalent to the claims.

Some or all of the subject matter described in the respective embodiments can be combined together. Some or all of the independent claims and their dependent claims described in the claims can be combined together, regardless of their dependent relationships. Furthermore, although a format (multiple dependent claim format) in which a claim dependent on two or more other claims is described is used in the claims, the claim form is not limited to this format. Claims may be described in a format (multi-multi dependent claim) in which a multiple dependent claim depends on at least one multiple dependent claim.

What is claimed is:

1. A medical device for determining a boundary in a blood vessel tomographic image, comprising:
a display;
a memory that stores a program; and
a processor configured to execute the program to perform the steps of:
acquiring data indicating a blood vessel tomographic image;
executing a call to a machine learning model with the data indicating the blood vessel tomographic image to detect a first region indicating a detection target and one or more second regions different from the first region, the machine learning model having been trained with:
a plurality of training blood vessel tomographic images, and
labels indicating positions of first regions and second regions different from the first regions for the training blood vessel tomographic images;
determining a boundary of the first region using the detected one or more second regions;
generating a screen showing the blood vessel tomographic image with a boundary line corresponding to the determined boundary; and
controlling the display to display the generated screen.

2. The medical device according to claim 1, wherein
the first region includes at least one of: a region indicating an inside of a stent, a region indicating a main trunk lumen of a blood vessel, a region indicating an inside of an external elastic membrane of a main trunk of the blood vessel, a region indicating an inside of an outer membrane of the main trunk of the blood vessel, a region indicating a side branch lumen of the blood vessel, a region indicating an inside of an external elastic membrane of a side branch of the blood vessel, a region indicating lumens of the main trunk and the side branch of the blood vessel, and a region indicating an inside of an external elastic membrane of the main trunk and the side branch of the blood vessel.

3. The medical device according to claim 1, wherein
the first region includes at least one of a plaque region, a thrombus region, a haematoma region, a calcified region, a dissection region, a crack region, an extravascular tissue region, and a device region.

4. The medical device according to claim 1, wherein
the one or more second regions include a region excluding the first region, and
the step of determining the boundary includes:
inverting the region excluding the first region, and
comparing the detected first region with the inverted region to determine the boundary.

5. The medical device according to claim 1, wherein
the first region indicates a side branch of a blood vessel,
one of the second regions is a region indicating a main trunk of the blood vessel, and another one of the second regions is a region indicating both the main trunk and the side branch, and
the step of determining the boundary includes:
excluding said one of the second regions from said another one of the second regions and specifying a region indicating the side branch, and
combining the specified region and the detected first region to determine the boundary.

6. The medical device according to claim 5, wherein
the steps further comprise determining a position of the side branch in the blood vessel tomographic image based on the determined boundary, and
the screen shows a marker at the determined position in the blood vessel tomographic image.

7. The medical device according to claim 1, wherein
the steps further comprise detecting a high brightness region or a low brightness region in the blood vessel tomographic image, and
the boundary is determined based on the high brightness region or the low brightness region.

8. The medical device according to claim 7, wherein
the first region includes a calcified region,
one of the second regions is a region indicating an inside of an external elastic membrane of a blood vessel, and another one of the second regions is a region indicating a lumen of the blood vessel, and
the step of determining the boundary includes:
specifying a region indicating a vessel wall based on said one of the second regions and said another one of the second regions,
detecting a high brightness region having brightness equal to or higher than a threshold in the specified region, and
combining the high brightness region and the first region to determine the boundary.

9. The medical device according to claim 8, wherein
the steps further comprise specifying an angle occupied by the calcified region in a circumferential direction of the blood vessel based on the determined boundary, and
the specified angle is indicated by an arc in the blood vessel tomographic image displayed on the screen.

10. The medical device according to claim 8, wherein the steps further comprise determining a feature of calcification based on a position of the calcified region with respect to a region indicating the vessel wall.

11. The medical device according to claim 7, wherein
the first region includes an attenuated plaque region,
the one or more second regions include a region indicating an inside of an external elastic membrane of a blood vessel,
the low brightness region is detected outside the region indicating the inside of the external elastic membrane, and
the step of determining the boundary includes combining the low brightness region and the attenuated plaque region.

12. The medical device according to claim 11, wherein
the steps further comprise specifying an angle occupied by the attenuated plaque region in a circumferential direction of the blood vessel based on the determined boundary, and
the specified angle is indicated by an arc in the blood vessel tomographic image displayed on the screen.

13. The medical device according to claim 1, wherein
the machine learning model detects, as among the first region and the one or more second regions, a plaque region and a region indicating a lumen of a blood vessel, and
the steps further comprise:
calculating a ratio of the plaque region to a cross section of the blood vessel at each position in an axial direction of the blood vessel based on an area of the plaque region and an area of the region indicating the lumen of the blood vessel in each of the blood vessel tomographic images continuously acquired in the axial direction, and
generating, from the continuously acquired blood vessel tomographic images, a longitudinal tomographic image showing a vessel wall in a different color depending on the calculated ratio.

14. The medical device according to claim 13, wherein
the machine learning model further detects, as among the first region and the one or more second regions, a region indicating a side branch of the blood vessel, and
the longitudinal tomographic image includes a predetermined marker at each position in the axial direction where the region indicating the side branch is detected.

15. A method for determining a boundary in a blood vessel tomographic image, the method comprising:
acquiring data indicating a blood vessel tomographic image;
executing a call to a machine learning model with the data indicating the blood vessel tomographic image to detect a first region indicating a detection target and one or more second regions different from the first region, the machine learning model having been trained with:
a plurality of training blood vessel tomographic images, and
labels indicating positions of first regions and second regions different from the first regions for the training blood vessel tomographic images;
determining a boundary of the first region using the detected one or more second regions; and
displaying a screen showing the blood vessel tomographic image with a boundary line corresponding to the determined boundary.

16. The method according to claim 15, wherein
the first region includes at least one of: a region indicating an inside of a stent, a region indicating a main trunk lumen of a blood vessel, a region indicating an inside of an external elastic membrane of a main trunk of the blood vessel, a region indicating an inside of an outer membrane of the main trunk of the blood vessel, a region indicating a side branch lumen of the blood vessel, a region indicating an inside of an external elastic membrane of a side branch of the blood vessel, a region indicating lumens of the main trunk and the side branch of the blood vessel, and a region indicating an inside of an external elastic membrane of the main trunk and the side branch of the blood vessel.

17. The method according to claim 15, wherein
the first region includes at least one of a plaque region, a thrombus region, a haematoma region, a calcified region, a dissection region, a crack region, an extravascular tissue region, and a device region.

18. The method according to claim 15, wherein
the one or more second regions include a region excluding the first region, and
the step of determining the boundary includes:
inverting the region excluding the first region, and
comparing the detected first region with the inverted region to determine the boundary.

19. The method according to claim 15, wherein
the first region indicates a side branch of a blood vessel,
one of the second regions is a region indicating a main trunk of the blood vessel, and another one of the second regions is a region indicating both the main trunk and the side branch, and
the step of determining the boundary includes:
excluding said one of the second regions from said another one of the second regions and specifying a region indicating the side branch, and
combining the specified region and the detected first region to determine the boundary.

20. A non-transitory computer-readable storage medium storing a program that causes a processor to perform a process comprising:
acquiring data indicating a blood vessel tomographic image;
executing a call to a machine learning model with the data indicating the blood vessel tomographic image to detect a first region indicating a detection target and one or more second regions different from the first region, the machine learning model having been trained with:
a plurality of training blood vessel tomographic images, and
labels indicating positions of first regions and second regions different from the first regions for the training blood vessel tomographic images;
determining a boundary of the first region using the detected one or more second regions; and
displaying a screen showing the blood vessel tomographic image with a boundary line corresponding to the determined boundary.
